(19) United States
(12) Patent Application Publication
(10) Pub. No.: US 2021/0186111 A1
(43) Pub. Date: Jun. 24, 2021

(54) APPARATUS FOR DETECTING SMOKE CONCENTRATION AND ELECTRONIC ATOMIZATION DEVICE

(71) Applicant: Shenzhen Smoore Technology Limited, Shenzhen (CN)

(72) Inventors: Qiang XU, Shenzhen (CN); Diansheng BAI, Shenzhen (CN); Huakai YUAN, Shenzhen (CN)

(21) Appl. No.: 17/100,953

(22) Filed: Nov. 23, 2020

(30) Foreign Application Priority Data
Dec. 20, 2019 (CN) 201911326063.5

(51) Int. Cl. A24F 40/53; G01N 15/06; A24F 40/51

(52) U.S. Cl. CPC A24F 40/53; G01N 15/06; A61M 15/06; G01N 2015/0693; A24F 40/51

(57) ABSTRACT

An apparatus for detecting smoke concentration includes a shell, a signal transmitter, and a signal receiver. The shell is arranged with an air channel tube, and the air channel tube defines an air channel. The signal transmitter and the signal receiver are arranged inside the shell. At least one of the signal transmitter and the signal receiver is arranged out of the air channel. The signal transmitter and the signal receiver work cooperatively to detect a concentration of the smoke in the air channel, so that the smoke cannot easily coagulate or adhere on the signal transmitter and/or the signal receiver, a signal is detected more accurately, and an error of a detection result is reduced.

### CROSS-REFERENCE TO RELATED APPLICATIONS

The present application claims the foreign priority of Chinese Patent Application No. 201911326063.5, filed on Dec. 20, 2019 in the National Intellectual Property Administration of China, the entire contents of which are hereby incorporated by reference in its entirety.

### TECHNICAL FIELD

The present disclosure relates to the field of detecting a smoke concentration, and in particular to an apparatus for detecting smoke concentration and an electronic atomization device.

### BACKGROUND

Detecting the mist of an atomized liquid may be an essential operation for various types of devices in the art. For example, when a user is using an inhaler for medical administration by inhalation, the medicine to be inhaled by the user may be quantified. User's health may be affected if an excessive amount of the medicine or an insufficient amount of the medicine is inhaled. Taking an electronic atomization device as another example, the user may experience a better taste when an amount of smoke after atomization is controlled appropriately. Controlling an amount of the smoke of the atomized liquid may be achieved by detecting a concentration of the smoke.

Concentration of the smoke of the atomized liquid may be detected by an optical signal transceiver, and the optical signal transceiver may directly contact the smoke. However, the smoke may coagulate on or adhere to the optical signal transceiver, thereby affecting the accuracy of the optical signal transceiver receiving an optical signal, such that a result of the detection may have a relatively large error.

### SUMMARY OF THE DISCLOSURE

According to a first aspect of the present disclosure, an apparatus for detecting smoke concentration includes a shell, an air channel tube, a signal transmitter and a signal receiver. The shell defines a receiving chamber. The air channel tube is received in the receiving chamber, and the air channel tube defines an air channel. The signal transmitter and the signal receiver are received in the receiving chamber. At least one of the signal transmitter and the signal receiver is arranged out of the air channel. The signal transmitter and the signal receiver are configured to work cooperatively to detect a concentration of smoke in the air channel.

According to a second aspect of the present disclosure, an apparatus for detecting smoke concentration includes a shell, a tube, and a signal transceiver assembly. The shell defines a receiving chamber. The tube is received in the receiving chamber and defines an air channel. Air and smoke may be capable of being received in and flow through the air channel. The signal transceiver assembly is received in the receiving chamber and includes a signal transmitter and a signal receiver. The signal transceiver assembly is arranged out of the air channel, and the signal transceiver assembly is configured to detect a concentration of the smoke in the air channel.

According to a third aspect of the present disclosure, an electronic atomization device includes an atomizer and an apparatus for detecting smoke concentration. The apparatus includes a shell, a tube, and a signal transceiver assembly. The shell defines a receiving chamber. The tube is received in the receiving chamber and defines an air channel. Air and smoke may be capable of being received in and flow through the air channel. The signal transceiver assembly is received in the receiving chamber and includes a signal transmitter and a signal receiver. The signal transceiver assembly is arranged out of the air channel, and the signal transceiver assembly is configured to detect a concentration of the smoke in the air channel. The atomizer is received in the receiving chamber and communicates with the air channel. The atomizer is configured to generate the smoke and transfer the smoke to the air channel.

### BRIEF DESCRIPTION OF THE DRAWINGS

In order to illustrate technical solutions of embodiments of the present disclosure, drawings required for illustrating the embodiments may be described in brief. Obviously, the following drawings show only some embodiments of the present disclosure. Other drawings may be obtained by any ordinary skilled person in the art without making creative work.

FIG. 1 is a perspective view of an apparatus for detecting smoke concentration according to an embodiment of the present disclosure.

FIG. 2 is a cross sectional view from a front of an apparatus for detecting smoke concentration according to an embodiment of the present disclosure.

FIG. 3 is an enlarged view of a portion of the apparatus for detecting smoke concentration shown in FIG. 2.

FIG. 4 is a cross sectional view from a side of an apparatus for detecting smoke concentration according to an embodiment of the present disclosure.

FIG. 5 is a cross sectional view from a front of an apparatus for detecting smoke concentration according to another embodiment of the present disclosure.

FIG. 6 is an enlarged view of a portion of the apparatus for detecting smoke concentration shown in FIG. 5.

FIG. 7 is a cross sectional view from a front of an apparatus for detecting smoke concentration according to another embodiment of the present disclosure.

FIG. 8 is an enlarged view of a portion of the apparatus for detecting smoke concentration shown in FIG. 7.

### DETAILED DESCRIPTION

Technical solutions of embodiments of the present disclosure may be clearly and comprehensively described by referring to the drawings. Obviously, the described embodiments are only some of, but not all of, the embodiments of the present disclosure. Any other embodiments obtained by an ordinary skilled person in the art based on the embodiments of the present disclosure without any creative work should also be within the scope of the present disclosure.

Terms of “first” and “second” in the present disclosure may be used for describing, but do not indicate or imply relevant significance of a feature or the number the feature. The term of “a plurality of” in the present disclosure refers to at least two, such as two, three, and the like, unless otherwise specified. Further, terms of “include”, “have”, and any form of the terms indicate non-exclusive inclusion. For example, a process, a method, a system, a product, or a device including a set of operations or units is not limited to the listed operations or units, but alternatively includes operations or units that not listed herein. Alternatively, other operations or units inherently included in the process, in the method, in the system, in the product, or in the device may be included. The term of “and/or” indicates a correlated relationship between objects, and three relationships may be included. For example, A and/or B may indicate three situations, i.e., only A is existed, A and B are existed at the same time, and only B is existed. Further, the character “/” in the present disclosure may indicate an object in front of the character “or” an object after the character.

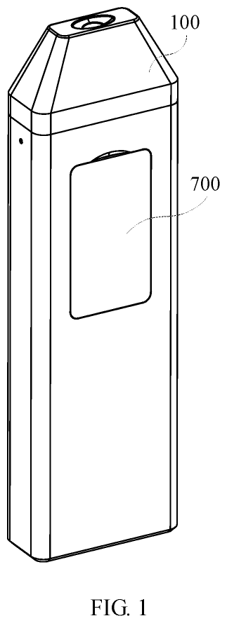

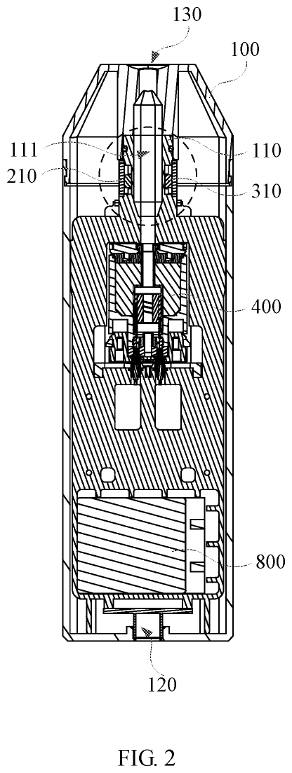

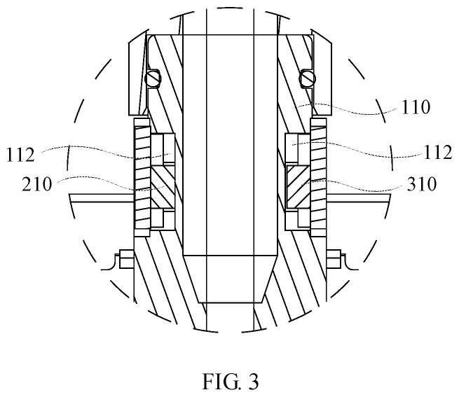

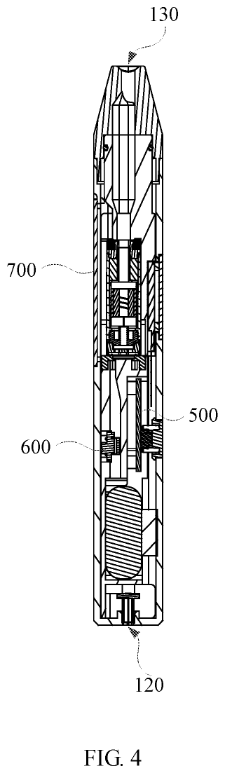

As shown in FIGS. 1 to 4, the apparatus for detecting smoke concentration is provided and includes a shell 100, a signal transmitter 210, and a signal receiver 310. The shell 100 may define a receiving chamber. An air channel tube 110 may be received in the receiving chamber and define an air channel 111. Smoke may be received in or flow through the air channel 111. The signal transmitter 210 and the signal receiver 310 may be received in the receiving chamber. At least one of the signal transmitter 210 and the signal receiver 310 may be arranged out of the air channel 111, i.e., may not be received in the air channel 111. In a process of detecting a concentration of smoke in the air channel 111, the signal transmitter 210 may be configured to generate a signal, and the signal receiver 310 may be configured to receive a signal. That is, the signal transmitter 210 and the signal receiver 310 may work cooperatively to detect the concentration of smoke received in the air channel 111.

According to the present disclosure, at least one of the signal transmitter 210 and the signal receiver 310 may not be received in the air channel 111 defined by the air channel tube 110, so that the smoke may not coagulate on or adhere to the signal transmitter 210 and/or the signal receiver 310, the signal may be detected more accurately, and an error of a detection result may be reduced.

Further, a recess 112 may be defined in the air channel tube 110. To be specific, the recess 112 may be defined by an outer surface of the air channel tube 110 being recessed inwardly. In the present embodiment, two recesses 112 may be opposite to each other. The signal transmitter 210 and the signal receiver 310 may be received in the two recesses 112, respectively. An exposed surface of the signal transmitter 210 and an exposed surface of the signal receiver 310 may align with the outer side of the air channel tube 110. A bottom wall of the recess 112 may be made of transparent material, such as transparent plastics, glass, and the like.

In other embodiments, the air channel tube 110 may be made of non-transparent material. The signal transmitter 210 and/or the signal receiver 310 may be arranged at an end of the air channel tube 110, such that at least a part of the signal transmitter 210 and/or the signal receiver 310 may not directly contact the smoke in the air channel tube 110. In this way, a reduced amount of smoke may coagulate or adhere to the signal transmitter 210 and/or the signal receiver 310.

In the present embodiment, the signal transmitter 210 and the signal receiver 310 may be arranged out of the air channel 111. The signal transmitter 210 may be an optical signal transmitter, and the signal receiver 310 may be an optical sensor. In other embodiments, the smoke concentration may be detected by image detection or by other means, which will not be limited by the present disclosure.

In the present embodiment, an angle between a signal transmission line of the signal transmitter 210 (not shown in the figure) and a signal receiving line of the signal receiver 310 may be in a range of 0° to 180°, such as 0°, 45°, or 180°. In other words, a first connection line may be between the signal transmitter 210 and a central axis of the air channel 111 along a signal transmission direction, and a connection line may be between the signal receiver 310 and the central axis of the air channel 111 along a signal receiving direction. An angle between the first connection line and the second connection line may be in a range of 0° to 180°, such as 0°, 45°, or 180°.

In the present embodiment, the angle between signal transmission line of the signal transmitter 210 and the signal receiving line of the signal receiver 310 may be 180°. The signal transmitter 210 and the signal receiver 310 may be disposed at two opposite sides of the air channel tube 111.

In the present embodiment, as the signal transmitter 210 and the signal receiver 310 are received in the recess 112, a thickness of the bottom wall of the recess may be less than a thickness of the other part of the air channel tube 110. In this way, an influence on the optical signal may be reduced, further improving accuracy of the detection.

In the present embodiment, at least a part of an inner side of the air channel tube 110 may be arranged with an isolation layer (not shown in the figure) to prevent the smoke from adhering. For example, an inner side of the wall of the recess 112 facing the air channel 111 may be arranged with the isolation layer. The isolation layer may be made of hydrophobic and lipophobic material. As the signal transmitter 210 and the signal receiver 310 may be precise elements, receiving the signal transmitter 210 and the signal receiver 310 in the air channel 111 and coating the hydrophobic and lipophobic isolation layer onto the signal transmitter 210 and the signal receiver 310 may significantly increase manufacturing costs. In the present embodiment, the isolation layer may be coated onto the inner side of the air channel tube 110 to reduce the manufacturing costs.

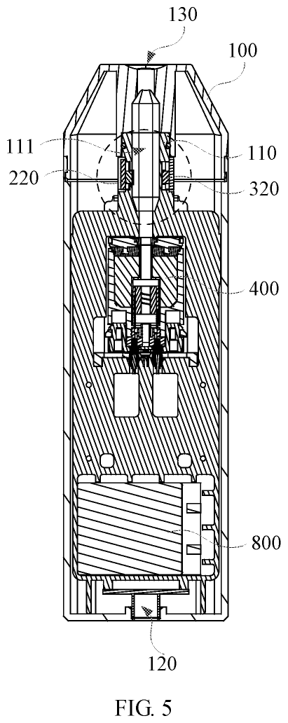

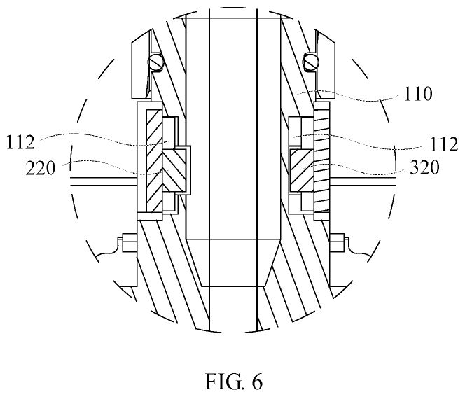

As shown in FIG. 5 and FIG. 6, an apparatus for detecting smoke concentration according to another embodiment of the present disclosure may include a shell 100, a signal transmitter 220 and a signal receiver 320. Structures of the shell 100, the signal transmitter 220 and the signal receiver 320 may be similar to structures of the shell, the signal transmitter and the signal receiver of the above embodiment, which will not be repeatedly described hereinafter. In the present embodiment, the signal transmitter 220 may be arranged to extend through the air channel tube 110 to directly contact the smoke in the air channel. The signal receiver 320 may not be received in the air channel 111, but received in the recess 112 in the air channel tube 110. The bottom wall of the recess 112 may be made of transparent material.

In other embodiments, the air channel tube 110 may further define a through hole along a thickness direction of the air channel tube. The signal transmitter 220 may not be received in the air channel 111, but received in the recess 112, whereas the signal receiver 320 may be arranged to extend through the through hole to directly contact the smoke in the air channel 111. The present disclosure does not limit which one of the signal transmitter 220 and the signal receiver 320 is received in the recess.

According to the apparatus for detecting smoke concentration provided in the present disclosure, the signal transmitter 210 or the signal receiver 310 may not be received in the air channel 111 defined by the air channel tube 110. At least one recess 112 may be defined on the outer side of the air channel tube 110. At least one of the signal transmitter 210 and the signal receiver 310 may be received in the at least one recess 112. The bottom wall of the recess 112 may be made of transparent material, such that the smoke may not easily coagulate or adhere to the signal transmitter 210 or the signal receiver 310. The signal may be detected more accurately, and the error of the detection result may be reduced.

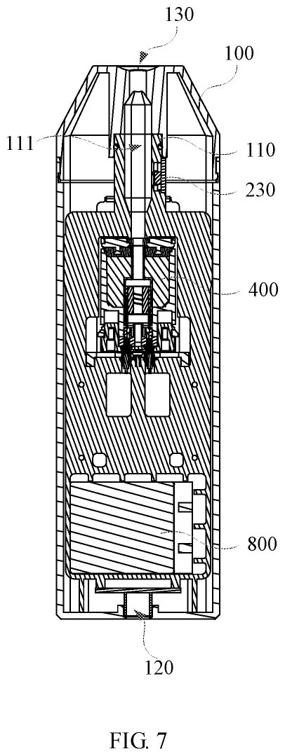

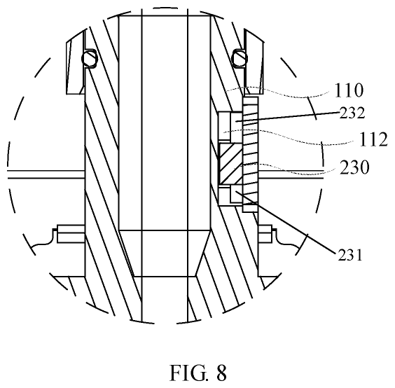

As shown in FIG. 7 and FIG. 8, an apparatus for detecting smoke concentration according to another embodiment of the present disclosure may include a shell 100 and a signal transceiver 230. A structure of the shell 100 may be similar to that in the above embodiment, which will not be repeatedly described hereinafter. In the present embodiment, the signal transceiver 230 may be referred to as one element integrating the signal transmitter 220 and the signal receiver 320. The signal transceiver 230 may include a signal transmission portion 231 and a signal receiving portion 232. In this way, the angle between a signal transmission line and a signal receiving line may be 0°. A recess 112 may be defined on an outer surface of the air channel tube 110. The signal transceiver 230 may be received in the recess 112, but may not be configured to extend through the air channel tube 110. A bottom wall of the recess 112 may be made of transparent material.

According to the present embodiment, the signal transceiver 230 may be arranged out of the air channel 111 defined by the air channel tube 110 of the shell 100, and the bottom wall of the recess 112 may be made of transparent material. In this way, the smoke may not easily coagulate or adhere to the signal transceiver 230, the signal may be detected more accurately, and the error of the detection result may be reduced.

As shown in FIGS. 1 to 4, an electronic atomization device may be provided and include an atomizer 400 and the apparatus for detecting smoke concentration. A structure of the apparatus for detecting smoke concentration is shown in the above embodiments and will not be repeatedly described hereinafter. The atomizer 400 may be arranged in the shell 100 and may communicate with the air channel 111, enabling generated smoke to flow to the air channel 111. The apparatus for detecting smoke concentration may be configured to detect the concentration of the smoke in the air channel 111.

In the present embodiment, the shell 100 further defines an air inlet 120 and an air outlet 130. The air may flow through the air inlet 120 to an inside of the shell 100. The air may further flow through the atomizer 400 to drive the smoke to flow through the air channel 111 and the air outlet 130 to reach an external of the device.

In the present embodiment, the electronic atomization device may further include a processor, an air flow meter 600, an indicator 700, and a battery 800. The processor 500, the air flow meter 600, and the battery 800 may be received in the receiving chamber defined by the shell 100. The indicator 700 may be arranged on the shell 100. The processor 500 may be connected to the apparatus for detecting smoke concentration, the atomizer 400, the air flow meter 600, the indicator 700, and the battery 800. The air flow meter 600 may be configured to detect a velocity of air flowing through the air inlet 120. The processor 500 may be configured to receive smoke concentration information sent from the apparatus for detecting smoke concentration and the velocity of air detected by the air flow meter 600. The indicator 700 may be configured to indicate an amount of smoke consumed by the atomizer or an amount of smoke remaining in the air channel.

In the present embodiment, the indicator 700 may be a display. In other embodiments, the indicator 700 may be an indicating lamp, a voice indicator, a vibrator, a signal transmitter, and the like, which will not be limited by the present disclosure.

In the present embodiment, the processor 500 may be configured to calculate the amount of smoke consumed at a particular time point or within a particular time range based on the smoke concentration information sent from the apparatus for detecting smoke concentration and the velocity of air detected by the air flow meter 600. In this way, an amount of smoke remaining in the air channel may be calculated based on the amount of smoke consumed at the particular time point or within the particular time range. Further, an amount smoke to be generated by the atomizer 400 may be adjusted based on the amount of smoke consumed at the particular time point or within the particular time range.

According to the apparatus for detecting smoke concentration provided in the embodiments of the present disclosure, at least one of the signal transmitter 210 and the signal receiver 310 may be arranged out of the air channel 111 defined by the air channel tube 110. The portion of the air channel tube 110 corresponding to the signal transmitter 210 and/or the signal receiver 310 may be made of transparent material. In this way, the smoke may not be easily coagulated or adhered to the signal transmitter 210 and/or the signal receiver 310, the signal may be detected accurately, and the error of the detection result may be reduced.

The above description illustrates implementations of the present disclosure, but does not limit the scope of the present disclosure. Any equivalent structural or process transformation performed based on the present drawings and the present specification, applied directly or indirectly in related art, should be within the scope of the present disclosure.

What is claimed is:

1. An apparatus for detecting smoke concentration, comprising:
a shell, defining a receiving chamber;
an air channel tube, received in the receiving chamber, wherein the air channel tube defines an air channel; and
a signal transmitter and a signal receiver, received in the receiving chamber, wherein at least one of the signal transmitter and the signal receiver is arranged out of the air channel, the signal transmitter and the signal receiver are configured to work cooperatively to detect a concentration of smoke in the air channel.

2. The apparatus according to claim 1, wherein a recess is defined on an outer surface of the air channel tube, at least one of the signal transmitter and the signal receiver is received in the recess, and a bottom wall of the recess is transparent.

3. The apparatus according to claim 1, wherein an angle between a signal transmission line of the signal transmitter and a signal receiving line of the signal receiver is in a range of 0° to 180°.

4. The apparatus according to claim 1, wherein at least a portion of an inner surface of the air channel tube corresponding to the signal transmitter and/or the signal receiver is arranged with an isolation layer.

5. The apparatus according to claim 4, wherein the isolation layer is a hydrophobic and lipophobic layer.

6. An apparatus for detecting smoke concentration, comprising:
a shell, defining a receiving chamber;
a tube, received in the receiving chamber and defining an air channel, wherein the air channel is configured to receive and conduct air and smoke; and
a signal transceiver assembly, received in the receiving chamber and comprising a signal transmitter and a signal receiver, wherein the signal transceiver assembly is arranged out of the air channel, and the signal transceiver assembly is configured to detect a concentration of the smoke in the air channel.

7. The apparatus according to claim 6, wherein a recess is defined on an outer surface of the tube, and the signal transceiver assembly is received in the recess.

8. The apparatus according to claim 7, wherein a bottom wall of the recess is transparent.

9. The apparatus according to claim 8, wherein an inner surface of the tube corresponding to the signal transceiver is covered by a hydrophobic and lipophobic layer.

10. The apparatus according to claim 6, wherein a first recess and a second recess are defined on an outer surface of the tube, the second recess is opposite to the first recess, the signal transmitter is received in the first recesses, and the signal receiver is received in the second recesses.

11. The apparatus according to claim 10, wherein an angle between a signal transmission line of the signal transmitter and a signal receiving line of the signal receiver is in a range of 0° to 180°.

12. The apparatus according to claim 6, wherein
a recess is defined on an outer surface of the tube;
the tube defines a through hole;
one of the signal transmitter and the signal receiver is configured to extend through the through hole; and
the other one of the signal transmitter and the signal receiver is received in the recess.

13. The apparatus according to claim 6, wherein
the signal transceiver assembly is arranged at an end of the tube; and
at least a part of the signal transceiver assembly is configured to contact the air and the smoke in the air channel.

14. An electronic atomization device, comprising an atomizer and an apparatus for detecting smoke concentration, wherein
the apparatus comprises:
a shell, defining a receiving chamber;
a tube, received in the receiving chamber and defining an air channel, wherein the air channel is configured to receive and conduct air and smoke; and
a signal transceiver assembly, received in the receiving chamber and comprising a signal transmitter and a signal receiver, wherein the signal transceiver assembly is arranged out of the air channel, and the signal transceiver assembly is configured to detect a concentration of the smoke in the air channel;
the atomizer is received in the receiving chamber and communicated with the air channel;
the atomizer is configured to generate the smoke and transfer the smoke to the air channel.

15. The electronic atomization device according to claim 14, wherein
the shell defines an air inlet and an air outlet;
the air is capable of flowing through the air inlet to an inside of the shell; and
the air is further capable of flowing through the atomizer to drive the smoke to flow through the air channel and the outlet to an outside of the electronic atomization device.

16. The electronic atomization device according to claim 15, further comprising a processor and an air flow meter, received in the receiving chamber, wherein
the processor is connected to the apparatus for detecting smoke concentration and the air flow meter;
the air flow meter is configured to detect a velocity of the air flowing through the air inlet; and
the processor is configured to receive smoke concentration information sent from the apparatus for detecting smoke concentration and the velocity of the air detected by the air flow meter.

17. The electronic atomization device according to claim 14, wherein a recess is defined on an outer surface of the tube, the signal transceiver is received in the recess, and a bottom wall of the recess is transparent.

18. The electronic atomization device according to claim 14, wherein an angle between a signal transmission line of the signal transmitter and a signal receiving line of the signal receiver is in a range of 0° to 180°.

19. The electronic atomization device according to claim 14, wherein at least a portion of an inner surface of the tube corresponding to the signal transceiver is covered by an isolation layer.

20. The electronic atomization device according to claim 19, wherein the isolation layer is a hydrophobic and lipophobic layer.
